(19) United States
(12) Patent Application Publication
(10) Pub. No.: US 2025/0195374 A1
(43) Pub. Date: Jun. 19, 2025

(54) COMPOSITIONS CONTAINING BIS-ETHYLHEXYLOXYPHENOL METHOXYPHENYL TRIAZINE AND AVOBENZONE

(71) Applicant: L'OREAL, Paris (FR)

(72) Inventors: Brian Bodnar, Manasquan, NJ (US); Sang Lee Bang, Jersey City, NJ (US); Martin Josso, Chevilly-Larue (FR); Anil Shah, East Windsor, NJ (US)

(21) Appl. No.: 18/543,756

(22) Filed: Dec. 18, 2023

(51) Int. Cl. A61K 8/49; A61K 8/35; A61K 8/37; A61Q 17/04

(52) U.S. Cl. CPC A61K 8/4966; A61K 8/35; A61K 8/37; A61Q 17/04; A61K 2800/51

(57) ABSTRACT

The present disclosure relates to compositions including 2,4-bis-{[4-(2-ethyl hexyloxy)-2-hydroxy]-phenyl}-6-(4-methoxyphenyl)-1,3,5-triazine, avobenzone, and optionally octinoxate, as well as to methods of making and using such compositions.

### FILED OF THE INVENTION

The present disclosure relates to compositions comprising 2,4-bis-{[4-(2-ethyl hexyloxy)-2-hydroxy]-phenyl}-6-(4-methoxyphenyl)-1,3,5-triazine (INCI name: bis-ethylhexyloxyphenol methoxyphenyl triazine), avobenzone and, optionally, octinoxate, as well as to methods of making and using such compositions.

### DISCUSSION OF BACKGROUND

Radiation of wavelengths between 290 nm and 400 nm allows tanning of the human epidermis, while radiation of wavelengths between 290 and 320 nm, called UVB rays, hinders the development of a natural tan. Exposure is also likely to lead to a detrimental change in the biomechanical properties of the epidermis, resulting in the appearance of wrinkles leading to premature aging of the skin (i.e. photoaging).

UVA rays with wavelengths between 320 and 400 nm penetrate deeper into the skin than UVB rays. UVA rays cause immediate and persistent browning of the skin. Daily exposure to UVA rays, even for a short time, under normal conditions can damage collagen fibers and elastin, resulting in a change in the microrelief of the skin, the appearance of wrinkles and uneven pigmentation (spots, lack of uniformity of complexion).

Many studies show the need for effective protection against UVA and UVB to prevent sunburn, photoaging, and the like.

In order to obtain a high protection product, it is generally necessary to combine a large number of sunscreens and/or a high amount of UV filters to achieve high levels of filtering efficiency.

However, high levels of UV filters do not lend themselves to an easy elaboration of compositions with a stabilized and pleasant texture.

Octinoxate (octyl methoxycinnamate) and avobenzone (4-tert-butyl-4′-methoxydibenzoylmethane) are UV filters which are not particularly stable against light, breaking down after exposure to light, which can result in less octinoxate and avobenzone being available over time and lower amounts of octinoxate and avobenzone available to effect their desired or intended protection against UV rays. Further, octinoxate has been reported to adversely affect avobenzone stability when present in the same composition as avobenzone, further affecting the amount and functionality of avobenzone present in the composition.

There remains a need in the art for improved consumer care compositions which possess good octinoxate and avobenzone stability against light and have pleasant application properties (e.g., non-greasy feeling).

Accordingly, one aspect of the present disclosure is compositions which possess good avobenzone stability against light and have pleasant application properties (e.g., non-greasy feeling).

Another aspect of the present disclosure is compositions which possess good octinoxate and avobenzone stability against light and have pleasant application properties (e.g., non-greasy feeling).

### SUMMARY OF THE INVENTION

The present disclosure relates to compositions comprising avobenzone and bis-ethylhexyloxyphenol methoxyphenyl triazine (BEMT or bemotrizinol). Preferably, the compositions further comprise octinoxate. Preferably, the bis-ethylhexyloxyphenol methoxyphenyl triazine (BEMT) is present in the compositions in an amount effective to improve avobenzone photostability against light, as well as to improve octinoxate photostability against light (if octinoxate is present).

The present disclosure also relates to methods of treating, caring for, protecting, enhancing the appearance of, and/or making up a keratinous material comprising applying compositions of the present disclosure to a keratinous material in an amount sufficient to treat, care for, enhance the appearance of, and/or make up the keratinous material.

The present disclosure also relates to methods of improving photostability of avobenzone and octinoxate (if present), in compositions comprising avobenzone and optionally octinoxate, wherein the methods comprise adding bis-ethylhexyloxyphenol methoxyphenyl triazine to the compositions during formation of the compositions in an amount sufficient to improve photostability of the avobenzone and octinoxate (if present).

The present disclosure also relates to methods of making compositions comprising combining bis-ethylhexyloxyphenol methoxyphenyl triazine, avobenzone, and optionally octinoxate in the compositions during formation of the compositions. Preferably, the bis-ethylhexyloxyphenol methoxyphenyl triazine (BEMT) is added to the compositions in an amount effective to improve photostability against light of the avobenzone and octinoxate (if present).

The present disclosure also relates to methods of improving avobenzone and optionally improving octinoxate stability against light of compositions comprising avobenzone and optionally octinoxate, wherein the methods comprise adding bis-ethylhexyloxyphenol methoxyphenyl triazine (BEMT) to the compositions during formation of the compositions in an amount sufficient to improve stability in the compositions against light of the avoboenzone and octinoxate (if present).

The present disclosure also relates to methods of improving avobenzone stability in compositions which also contain octinoxate, wherein the methods comprise adding bis-ethylhexyloxyphenol methoxyphenyl triazine (BEMT) to the compositions during formation of the compositions in an amount sufficient to improve avobenzone stability in the compositions which also contain octinoxate.

It is to be understood that both the foregoing general description and the following detailed description are exemplary and explanatory only, and are not restrictive of the disclosure.

### DETAILED DESCRIPTION OF THE INVENTION

In the following description and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5 includes specifically the integers within the range 1, 2, 3, 4 and 5, as well as subranges such as and 2-5, 3-5, 2-3, 2-4, 1-4, etc., as well as all fractional numbers within the range such as 1.2, 2.3, 3.4, etc., and subranges including such fractional numbers such as 1.5-3.8, 2-4.3, 4.2-4.9, etc.

“Film former”, “film-forming polymer” or “film-forming agent” as used herein means a polymer or resin which is capable of leaving a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as hydrogen atoms or chlorine atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the disclosure provided that these amounts do not materially affect at least one, preferably most, of the advantageous properties of the compositions of the disclosure. Thus, for example, “free of oil” means that an effective amount (that is, more than trace amounts) of oil(s) is/are omitted from the composition (that is, about 0% by weight), “substantially free of oil” means that oil(s) is/are present in amounts not greater than 0.1% by weight, and “devoid of oil” means that oil(s) is/are present in amounts not greater than 0.25% by weight, based on the total weight of the composition. The same nomenclature applies for all other ingredients identified throughout the application and in this paragraph such as, for example, specific UV filters and/or surfactants (compositions of the disclosure which are “free of oxybenzone,” “substantially free of oxybenzone,” and “devoid of oxybenzone,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes organic UV filters such as avobenzone, octocrylene, benzophenones, benzotriazoles and merocyanines, as well as mineral UV filters such as zinc oxide (ZnO) and titanium dioxide (TiO2).

“Anhydrous” as it is used herein means that compositions of the disclosure contain less than 3% water, meaning that the compositions can also contain less than 2% water, and less than 1% water, as well as being “free of water,” “substantially free of water,” and “devoid of water” as defined above.

“Keratinous materials” means nails (finger and/or toe nails), skin such as body, face, and eye area, scalp, keratin fibers such as eyelashes, eyebrows, and hair, and mucous membranes such as lips.

“Physiologically acceptable” means compatible with keratinous materials and having a pleasant color, odor and feel, and which does not cause any unacceptable discomfort (stinging or tautness) liable to discourage a consumer from using the composition.

“UV protection efficiency” or “filtering efficiency” in the context of the present disclosure, Is evaluated from one or more of SPF, UVAPF, Critical Wavelength, and UVA-V/UV ratio.

“SPF” (Sun Protection Factor) measures the level of protection against erythema provided by a composition. The SPF value corresponds to the ratio between the minimum erythemal dosage (MED) measured wearing the composition and the MED measured with bare skin. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989).

The evaluation of the SPF (Sun Protection Factor) can be performed, for example, in vitro with spectrophotometer by Labsphere (North Sutton, NH, USA).

In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the Sun Protection Factor (SPF) can also be performed in-vivo according to the ISO 24444:2019 protocol “Cosmetics-Sun protection test methods-In-vivo determination of the sun protection factor (SPF).” Also, it can be determined according to FDA protocols, as described in the document “Labeling and Effectiveness Testing; Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

The evaluation of UVA protection can also be measured in vitro with the Labsphere® spectrophotometer under conditions such as those discussed above in connection with SPF. ISO 24443:2021 protocol describes such an in vitro method.

FDA broad spectrum testing procedures, in particular “critical wavelength” testing procedures, can also be found at 21 C.F.R. Part 352 Subpart D § 352.72. Also, broad spectrum testing procedures include determining the UVA1/UV ratio as described in “Sunscreen Drug Products for Over-the-Counter Human Use” published in the Federal Register https://www.federalregister.gov/documents/2019/02/26/2019-03019/sunscreen-drug-products-for-over-the-counter-human-use.” In the assay outlined in the monograph, known as the Boots adaptation of the Diffey/Robson test method, a ratio is generated of the protection afforded by the sunscreen product from UVA1 (340-400 nm) compared to the protection from total UV radiation (UVB and UVA at 290-400 nm) calculated from the absorbance curve. This ratio UVA1/UV would represent the score for the product in the in vitro test.

According to the present disclosure, compositions of the present disclosure preferably have one or more of the following properties:

Compositions have a critical wavelength as determined by FDA's critical wavelength procedures of at least 370 nm;

Compositions have an SPF value of at least 15, preferably at least 30, preferably at least 50 and preferably at least 70;

Compositions have a UVAPF/SPF ratio of at least 1/3, and preferably at least 2/5; and/or

Compositions have a UVA1/UV ratio of 0.7 or higher, preferably 0.75 or higher, and preferably 0.8 or higher.

“Makeup Result” as used herein relates to the visual impact of a composition when applied to a keratinous material such as the skin. The makeup result may relate to the apparent color of the product after application on a keratinous material or an optical effect such as shine, blurring or fine lines and imperfections, or hiding effect on pores or blemishes after the product is applied to the keratinous material.

“Long wear” as used herein, refers to compositions where color or other apparent properties remains the same or substantially the same as at the time of application, as viewed by the naked eye, after an extended period of time or after a particular stress event such as water immersion or rubbing. “Long wear” may be evaluated by evaluating long wear properties by any method known in the art for evaluating such properties. For example, long wear may be evaluated by a test involving the application of a composition to keratinous material such as skin and evaluating the color of the composition after an extended period of time. For example, the color of a composition may be evaluated immediately following application to keratinous material such as skin and these characteristics may then be re-evaluated and compared after a certain amount of time. Further, these characteristics may be evaluated with respect to other compositions, such as commercially available compositions. “Long wear” may also be evaluated using vitro methods on non-keratinous substrates such as polymethylmethacrylate (PMMA), wherein properties such as color, transparency, or vitro SPF may be evaluated before and after a period of time or stressor such as water immersion, incubation at elevated temperatures, or an abrasive technique is applied.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means about 20-25° C.

“Atmospheric pressure” means about 760 mmHg, i.e. about 105 pascals.

“UV filter” and “sunscreen agent” are used interchangeably in this application.

“UV efficacy”, “UV protection”, and “UV efficiency” are used interchangeably in this application.

“Photostability” and “stability against light” are used interchangeably in this application.

The compositions and methods of the present disclosure can comprise, consist of, or consist essentially of the essential elements and limitations of the disclosure described herein, as well as any additional or optional ingredients, components, or limitations described herein or otherwise useful. For example, the UV (ultraviolet) absorbing system of the compositions of the disclosure can “consist essentially of” bis-ethylhexyloxyphenol methoxyphenyl triazine and avobenzone alone, or bis-ethylhexyloxyphenol methoxyphenyl triazine, avobenzone and octinoxate alone, or bis-ethylhexyloxyphenol methoxyphenyl triazine, avobenzone and optionally octinoxate in combination with oxides of zinc, titanium and/or cerium, and/or one or more additional organic UV filters.

Similarly, the avobenzone and octinoxate (if present) protection against light system (“avobenzone and octinoxate (if present) protection system”) of the compositions of the disclosure can “consist essentially of” bis-ethylhexyloxyphenol methoxyphenyl triazine. If octinoxate is not present in the compositions, it can be referred to as the “avobenzone protection system”).

Similarly, the avobenzone protection against degradation caused by octinoxate system (“avobenzone degradation protection system”) of the compositions of the disclosure can “consist essentially of” bis-ethylhexyloxyphenol methoxyphenyl triazine.

For purposes of the present disclosure, the “basic and novel property” associated with compositions, components and methods related to avobenzone (and optionally octinoxate) which “consist essentially of” identified ingredients or actions is “avobenzone (and optionally octinoxate) stability against light.”

For purposes of the present disclosure, the “basic and novel property” associated with compositions, components and methods related to avobenzone which “consist essentially of” identified ingredients or actions is “avobenzone stability against light and degradation caused by octinoxate.”

“Avobenzone stability against light” and ““avobenzone stability against light and degradation caused by octinoxate” as used herein means stability against degradation over time after exposure to UV radiation is limited such that the amount of avobenzone (optionally octinoxate) available for application has substantially remained constant over the time period. Avobenzone (optionally octinoxate) stability can be assessed by determining amount of avobenzone (optionally octinoxate) present in a composition at an initial time (time=T₀) and comparing this determination with a determination of amount of avobenzone (optionally octinoxate) present in the composition at a later date after exposure of the composition to a pre-determined UV dose. Avobenzone (optionally octinoxate) stability may also be assessed by comparing the absorption spectra before and after a composition comprising avobenzone (optionally octinoxate) is irradiated with a pre-determined amount of UV light. Irradiation of any composition comprising avobenzone (optionally octinoxate) may be performed using a single wavelength, preferably the peak absorbance in the UV region for the UV filter (lambda max), or over a range of wavelengths as provided by a Solar Simulator or otherwise similar means of irradiation. Where a comparison of absorption spectra are used for assessment of avobenzone (optionally octinoxate) stability, it may be convenient to compare the vitro SPF values measured before and after irradiation under either single wavelength or a range of wavelengths of UV light.

By “substantially remained constant,” it is meant that at least 70% of target compound has remained in the composition, preferably at least 80%, preferably at least 85%, and preferably at least 90%, over the time period. Where in vitro SPF or other calculated measurements are used to assess the target compound stability, “substantially remained constant” may be understood that at least 70% of the in vitro SPF is maintained for the composition after irradiation, preferably at least 80%, preferably at least 85%, and preferably at least 90%, compared to the in vitro SPF before irradiation.

Compositions of the present disclosure may be in any form suitable for use as a personal care composition, such as that of a stick, a paste, a cream, an anhydrous composition, an emulsion (oil-in-water, water-in-oil, multiple emulsion such as oil-in-water-in-oil), nanoemulsion, a gel, a liquid, a solid, etc. These compositions can be used for any personal care purpose in cosmetic and/or dermatological products such as, for example, a sunscreen, a foundation, lip balms, lipsticks, concealers, mascaras, leave-in hair products, eye shadows, powders, etc.

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Composition

### Bis-Ethylhexyloxyphenol Methoxyphenyl Triazine, Avobenzone and Octinoxate

According to the present disclosure, compositions comprising a UV (ultraviolet) absorbing system comprising 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), avobenzone and optionally octinoxate are provided.

According to preferred embodiments, the UV absorbing system comprises at least 5% by weight of the bis-ethylhexyloxyphenol methoxyphenyl triazine, preferably at least 15% by weight of the bis-ethylhexyloxyphenol methoxyphenyl triazine, preferably at least 20% by weight of the bis-ethylhexyloxyphenol methoxyphenyl triazine, preferably at least 30% by weight of the bis-ethylhexyloxyphenol methoxyphenyl triazine, preferably at least 40% by weight of the bis-ethylhexyloxyphenol methoxyphenyl triazine, preferably at least 50% by weight of the bis-ethylhexyloxyphenol methoxyphenyl triazine, preferably at least 70% by weight of the bis-ethylhexyloxyphenol methoxyphenyl triazine, preferably at least 80% by weight of the bis-ethylhexyloxyphenol methoxyphenyl triazine, and preferably at least 90% by weight of the bis-ethylhexyloxyphenol methoxyphenyl triazine, with all weights being based on the total weight of the UV absorbing system.

According to preferred embodiments, the UV absorbing system comprises at least 5% by weight of the avobenzone, preferably at least 15% by weight of the avobenzone, preferably at least 20% by weight of the avobenzone, preferably at least 30% by weight of the avobenzone, preferably at least 40% by weight of the avobenzone, preferably at least 50% by weight of the avobenzone, preferably at least 70% by weight of the avobenzone, preferably at least 80% by weight of the avobenzone, and preferably at least 90% by weight of the avobenzone, with all weights being based on the total weight of the UV absorbing system.

According to preferred embodiments, the UV absorbing system optionally comprises at least 5% by weight of the octinoxate, preferably at least 15% by weight of the octinoxate, preferably at least 20% by weight of the octinoxate, preferably at least 30% by weight of the octinoxate, preferably at least 40% by weight of the octinoxate, preferably at least 50% by weight of the octinoxate, preferably at least 70% by weight of the octinoxate, preferably at least 80% by weight of the octinoxate, and preferably at least 90% by weight of the octinoxate, with all weights being based on the total weight of the UV absorbing system. “UV absorbing system” contains all organic UV filters and/or mineral UV filters present in the composition.

According to preferred embodiments, bis-ethylhexyloxyphenol methoxyphenyl triazine is present in compositions of the present disclosure in a UV absorbing effective amount such as, for example, from about 0.1% to about 10% by weight with respect to the total weight of the composition, from about 1% to about 10% by weight, from about 2.5% to about 8% by weight, and from about 4% to about 6% by weight, including all ranges and subranges therebetween such as, for example, from about 4.5% to about 10% by weight, and from about 1% to about 8% by weight, from about 1% to about 6% by weight, from about 5.5% to about 8% by weight, from about 5.7% to about 9% by weight, etc., with all weights being based on the total weight of the composition.

According to preferred embodiments, avobenzone is present in compositions of the present disclosure in a UV absorbing effective amount such as, for example, from about 0.1% to about 5% by weight with respect to the total weight of the composition, from about 0.5% to about 4% by weight, from about 1% to about 3.5% by weight, and from about 2% to about 3% by weight, including all ranges and subranges therebetween such as, for example, from about 1.5% to about 3% by weight, from about 1% to about 2% by weight, from about 0.25% to about 4% by weight, from about 2.5% to about 4.5% by weight, from about 1.7% to about 3.9% by weight, etc., with all weights being based on the total weight of the composition.

According to preferred embodiments, octinoxate is optionally present in compositions of the present disclosure in a UV absorbing effective amount such as, for example, from about 0.1% to about 10% by weight with respect to the total weight of the composition, from about 1% to about 10% by weight, from about 1% to about 7.5% by weight, and from about 2% to about 6% by weight, including all ranges and subranges therebetween such as, for example, from about 4.5% to about 10% by weight, and from about 1% to about 8% by weight, from about 1% to about 6% by weight, from about 2.5% to about 7.5% by weight, from about 5.7% to about 9% by weight, etc., with all weights being based on the total weight of the composition.

According to preferred embodiments, bis-ethylhexyloxyphenol methoxyphenyl triazine is present in compositions of the present disclosure in an avobenzone stability against UV rays and avobenzone stability against UV rays effective amount such as, for example, from about 0.1% to about 10% by weight with respect to the total weight of the composition, from about 1% to about 10% by weight, from about 2.5% to about 8% by weight, and from about 4% to about 6% by weight, including all ranges and subranges therebetween such as, for example, from about 4.5% to about 10% by weight, and from about 1% to about 8% by weight, from about 1% to about 6% by weight, from about 5.5% to about 8% by weight, from about 5.7% to about 9% by weight, etc., with all weights being based on the total weight of the composition. Typically, the amount of avobenzone (and optionally octinoxate) stability against UV rays of the avobenzone (and optionally octinoxate) provided by bis-ethylhexyloxyphenol methoxyphenyl triazine can be affected by the relative amounts of the octinoxate (if present), avobenzone and bis-ethylhexyloxyphenol methoxyphenyl triazine present in the composition. The more bis-ethylhexyloxyphenol methoxyphenyl triazine present in a composition, the greater the octinoxate (if present) and avobenzone stability of the octinoxate (if present) and avobenzone. Also, the less octinoxate (if present) and avobenzone present in the composition, the greater the octinoxate (if present) and avobenzone stability, particularly in embodiments in which higher amounts of bis-ethylhexyloxyphenol methoxyphenyl triazine are present in the composition.

According to preferred embodiments, bis-ethylhexyloxyphenol methoxyphenyl triazine and the octinoxate (if present) are present in compositions of the present disclosure in a weight % ratio of preferably about 10:1 to about 1:10, preferably about 7.5:1 to about 1:7.5, preferably about 5:1 to about 1:5, and preferably about 2:1 to about 1:2, including all ranges and subranges therebetween, such as for example 10:1 to 2.5:1, 7.5:1 to 1:1, 6:1 to 1.5:1, 1:1 to 1:8, 1:1.5 to 1:8, etc, with all weights being based on the weight % of bis-ethylhexyloxyphenol methoxyphenyl triazine and weight % of the octinoxate present in the composition. Preferably, a higher weight % of the bis-ethylhexyloxyphenol methoxyphenyl triazine is present in the compositions than of the octinoxate (if present).

According to preferred embodiments, bis-ethylhexyloxyphenol methoxyphenyl triazine and the avobenzone are present in compositions of the present disclosure in a weight % ratio of preferably about 25:1 to about 1:25, preferably about 20:1 to about 1:20, preferably about 101 to about 1:10, and preferably about 3:1 to about 1:3, including all ranges and subranges therebetween, such as for example 15:1 to 2.5:1, 7.5:1 to 1:1, 6:1 to 1.5:1, 1:1 to 1:3, 1:2.5 to 1:5, etc, with all weights being based on the weight % of bis-ethylhexyloxyphenol methoxyphenyl triazine and weight % of the avobenzone present in the composition. Preferably, a higher weight % of the bis-ethylhexyloxyphenol methoxyphenyl triazine is present in the compositions than of the avobenzone.

### Additional Sunscreen Agents

According to preferred embodiments of the present disclosure, compositions optionally further comprising at least one additional UV filter (in addition to bis-ethylhexyloxyphenol methoxyphenyl triazine, avobenzone and octinoxate) selected from the group consisting of organic UV filters, oxides of zinc, oxides of cerium, oxides of titanium, and mixtures thereof are provided.

Additional organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Suitable organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds different from Bis-Ethylhexyloxyphenol Methoxyphenyl Triazine; benzotriazole compounds; benzalmalonate compounds including those mentioned in patent U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from—alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. Preferably, the lipophilic organic UV filters are selected from salicylic compounds, dibenzoylmethane compounds, benzylidene camphor compounds; benzophenone compounds; triazine compounds different from Bis-Ethylhexyloxyphenol Methoxyphenyl Triazine; benzotriazole compounds; as well as other categories of compounds identified herein; and mixtures thereof.

Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β,β-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Hellopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Hellopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxybenzoyl)benzoate (such as that marketed under the tradename UVINUL A+ by BASF).

Examples of triazine compounds include Diethylhexyl butamido triazone, such as that marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzalmalonate compounds include Dineopentyl 4′-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669,323 and U.S. Pat. No. 2,463,264.

Suitable para-aminobenzoic acid compounds include PABA (p-aminobenzoic acid), ethyl PABA, Ethyl dihydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis [6-(2H-benzotriazol-2-yl)-4-methyl-phenol], such as that marketed under the trademark “Mixxim BB/200” by Fairmount Chemical, 2,2′-methylenebis [6-(2H-benzotriazol-2-yl)-4-(I,I,3,3-tetramethylbutyl) phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or—“Mexoryl XL” by L′Oréal.

Examples of benzoxazole compounds include 2,4-bis [5-I(dimethylpropyl)benzoxazol-2-yl-(4-phenyl)imino]-6-(2-ethylhexyl)imino-I,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include I,I-dicarboxy (2,2′-dimethylpropyl)-4,4-diphenylbutadiene.

According to preferred embodiments, compositions of the present disclosure further comprise at least one additional organic UV filter selected from the group consisting of Octisalate (ethylhexyl salicylate), Ensulizole (Phenylbenzimidazole sulfonic acid), Homosalate, Octocrylene, and mixtures thereof. In such embodiments, the UV absorbing system can “consist of” or “consist essentially of” (1) bis-ethylhexyloxyphenol methoxyphenyl triazine, (2) avobenzone, (3) optionally octinoxate, and (4) at least one organic UV filter selected from the group consisting of Octisalate (ethylhexyl salicylate), Ensulizole (Phenylbenzimidazole sulfonic acid), Homosalate, Octocrylene, and mixtures thereof.

According to other preferred embodiments, however, compositions of the present disclosure are “free of,” “substantially free of,” or “devoid of” as defined above of one or more of additional organic UV filters selected from the group consisting of Octisalate (ethylhexyl salicylate), Ensulizole (Phenylbenzimidazole sulfonic acid), Homosalate, and Octocrylene, preferably two or more, preferably three or more, or preferably all four of these sunscreen agents.

According to preferred embodiments, compositions of the present disclosure are “free of,” “substantially free of,” or “devoid of” as defined above of one or more of additional organic UV filters selected from the group consisting of OXYBENZONE (benzophenone-3), ETHYLHEXYL TRIAZONE, DROMETRIZOLE TRISILOXANE, METHYLENE BIS-BENZOTRIAZOLYL TETRAMETHYLBUTYL PHENOL, DIETHYLAMINO HYDROXY BENZOYL HEXYL BENZOATE, DIETHYLHEXYL BUTAMIDO TRIAZONE, ISOAMYL P-METHOXYCINNAMATE, POLYSILICONE-15, 4-METHYLBENZYLIDENE CAMPHOR, DISODIUM PHENYL DIBENZIMIDAZOLE TETRASULFONATE, METHOXYPROPYLAMINO CYCLOHEXENYLIDENE ETHOXYETHYLCYANOACET ATE, preferably two or more, preferably three or more, preferably four or more, etc., and preferably “free of,” “substantially free of,” or “devoid of” all of these sunscreen agents.

According to preferred embodiments, compositions of the present disclosure are “free of,” “substantially free of,” or “devoid of” as defined above of OXYBENZONE (benzophenone-3).

If present in compositions of the present disclosure, the at least one additional UV filter is preferably present in compositions of the present disclosure in an amount of at least about 5% by weight, preferably at least about 10% by weight, preferably at least about 12% by weight, preferably at least about 14% by weight, and preferably at least about 15% by weight, with the upper end of the range of additional UV filter present preferably being about 40% by weight (e.g., about 5-40%, about 10-40%, about 12-40%, etc.), preferably about 30% by weight (e.g., about 5-30%, about 10-30%, about 12-30%, etc.), preferably about 25% by weight (e.g., about 5-25%, about 10-25%, about 12-25%, etc.), and preferably about 20% by weight (e.g., about 5-20%, about 10-20%, about 12-20%, etc.), with all weights being based on the total weight of the composition.

According to preferred embodiments, compositions of the present disclosure comprise 10% or less by weight relative to the total weight of composition of additional UV filters, preferably less than 5% by weight relative to the total weight of composition, preferably less than 3% by weight relative to the total weight of composition, and preferably less than 1% by weight relative to the total weight of composition.

According to preferred embodiments, compositions of the present disclosure comprise 10% or less by weight relative to the total weight of composition of additional organic UV filters, preferably less than 5% by weight relative to the total weight of composition, preferably less than 3% by weight relative to the total weight of composition, and preferably less than 1% by weight relative to the total weight of composition.

According to preferred embodiments, the UV absorbing system and/or octinoxate protection system and/or avobenzone protection system of compositions of the present disclosure can “consist of” or “consist essentially of” bis-ethylhexyloxyphenol methoxyphenyl triazine, avobenzone, and optionally octinoxate.

According to preferred embodiments, the UV absorbing system and/or octinoxate protection system and/or avobenzone protection system of compositions of the present disclosure can “consist of” or “consist essentially of” (1) bis-ethylhexyloxyphenol methoxyphenyl triazine, (2) avobenzone, (3) optionally octinoxate, and (4) oxides of zinc, oxides of titanium and/or oxides of cerium.

According to preferred embodiments, the UV absorbing system and/or octinoxate protection system and/or avobenzone protection system of compositions of the present disclosure can “consist of” or “consist essentially of” (1) bis-ethylhexyloxyphenol methoxyphenyl triazine, (2) avobenzone, (3) optionally octinoxate, and (4) additional organic UV filter(s).

According to preferred embodiments, this disclosure contemplates omitting one or more of any of the specific UV filters discussed above from the UV absorbing system and/or octinoxate protection system and/or avobenzone protection system of compositions of the present disclosure. By way of example, octocrylene can be omitted from the compositions. Similar omission of one or more of any of the specific UV filters discussed is thus contemplated.

### Oil

According to preferred embodiments of the present disclosure, compositions further comprising at least one oil are provided. “Oil” means a substance which is hydrophobic and lipophilic, and is a liquid at about room temperature (20 to 25° C.) and about atmospheric pressure (760 mm Hg).

Suitable include volatile and/or non-volatile oils. Such oils can be any acceptable oil including but not limited to silicone oils and/or hydrocarbon oils.

According to certain embodiments, the compositions of the present disclosure preferably comprise one or more volatile silicone oils. Examples of such volatile silicone oils include linear or cyclic silicone oils having from 2 to 7 silicon atoms, these silicones being optionally substituted with alkyl or alkoxy groups of 1 to 10 carbon atoms. Specific oils that may be used in the disclosure include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present disclosure. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present disclosure, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

According to certain embodiments of the present disclosure, the composition comprises at least one non-volatile oil. Examples of non-volatile oils that may be used in the present disclosure include, but are not limited to, polar oils such as, for example:
esters and ethers, in particular fatty acids, such as oils of formulas R1COOR2, wherein R 1 represents the remainder of a fatty acid having from 8 to 29 carbon atoms, and R 2 represents a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, fatty alcohol decanoates; polyol esters, such as propylene glycol dioctanoate, neopentylglycol diheptanoate and diethylene glycol diisononanoate; tryiglycerides such as caprylic/capric acid triglycerides, for instance those sold by the company Stearineries Dubois or those sold under the names Miglyol 810, 812 and 818 by the company Dynamit Nobel; and esters of pentaerythritol such as pentaerythrityl tetraisostearate or dipentaerythrityl pentaisononanoate;
ethers containing from 10 to 40 carbon atoms;
liquid C₈ to C₂₆ fatty alcohols, for instance oleyl alcohol, cetyl alcohol, stearyl alcohol, octyldodecanol, and cetearly alcohol;
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or, for example, sunflower, corn, soybean, squash, grape seed, sesame, hazelnut, apricot, macadamia, ara, sunflower oil, castor oil, avocado, triglycerides of caprylic/capric acids such as those sold by the company Stearineries Dubois or those sold under the names Miglyol 810, 812 and 818 by the company Dynamit Nobel, coco-caprylate/caprate (esterified oil from coconut oil), jojoba oil, shea butter oil; and
mixtures thereof.

Further, examples of non-volatile oils that may be used in the present disclosure include, but are not limited to, non-polar oils such as branched and unbranched hydrocarbons, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present disclosure, the compositions of the present disclosure comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Particularly preferred oils include, but are not limited to, one or more of the following: Diisopropyl Sebacate, C12-15 Alkyl Benzoate, Phenethyl Benzoate, Isopropyl Lauroyl Sarcosinate, Diisopropyl Adipate, Dibutyl Adipate, Dicaprylyl Carbonate, Dicaprylate/Dicaprate, Coco Glycerides, Caprylic/capric triglyceride, Isopropyl Myristat, Isopropyl Palmitate, Coco Caprylate/Caprate, Ethylhexyl Palmitate, Isononyl isononanoate, Octyl dodecanol, Isohexadecane, isododecane, Dicaprylyl Ether, C15-19 Alkane, and mixtures thereof.

According to preferred embodiments, the at least one oil is/are present in the compositions of the present disclosure in an amount ranging from about 1% to about 50% by weight, more preferably from about 5 to about 40% by weight, and preferably from about 10% to about 35% by weight, based on the total weight of the composition, including all ranges and subranges within these ranges such as, for example, 15% to 40%, 20% to 45%, etc.

### Aqueous Phase

The compositions of the present disclosure may also optionally contain water. When the compositions of the present disclosure contain water, they are preferably in the form of an emulsion. Preferably, when the compositions of the present disclosure contain water, they are in the form of an emulsion containing an external aqueous phase such as an oil-in-water emulsion (O/W) or a water-in-oil-in-water emulsion (O/W/O), or an emulsion containing an external oil phase such as a water-in-oil emulsion (W/O) or an oil-in-water-in-oil (O/W/O) emulsion. Preferably, when in the form of an emulsion, the oil phase can contain silicone oils (e.g., Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

According to preferred embodiments, however, compositions of the present disclosure are devoid of water, substantially free of water, or free of water as defined herein. Preferably, the compositions of the present disclosure are anhydrous.

If present in compositions of the present disclosure, the aqueous phase may comprise at least one water-soluble organic solvent which is liquid at room temperature and atmospheric pressure. For example, such at least one water-soluble organic solvent may include:

Triethyl citrate;

C1-C5 monoalcohols having a C1-C5 alkane chain and a single hydroxyl function (OH). Suitable C1-C5 monoalcohols include methanol, ethanol, propanol, isopropanol, butanol and mixtures thereof;

polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;

and mixtures thereof.

According to preferred embodiments, the at least one water-soluble organic solvent is selected from the group consisting of ethanol, dipropylene glycol, butylene glycol, propanediol and propylene glycol, and mixtures thereof.

If present, the water-soluble organic solvent(s) is/are preferably present in compositions of the present disclosure in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

### Additional Optional Ingredients

Compositions of the present disclosure may also optionally further include at least one additive or auxiliary commonly used in cosmetic compositions and known to a person skilled in the art as being capable of being incorporated into such compositions. Such additives or auxiliaries may be chosen from antioxidants, film formers, colorants (e.g., pigments and dyes), waxes, active agents, active agent protection compounds against light such as piperidinol compounds, SPF boosters such as diethylhexyl syringylidenemalonate, ethylhexyl methoxycrylene and butyl octyl salicylate, thixotropic agents (e.g., clays), fillers, preservatives, fragrances, surfactants, spreading agents, dispersing agents, antifoaming agents, neutralizing agents, stabilizing agents, fibers, wetting agents and their mixtures. Although, preferably, compositions of the present disclosure are “free of,” “substantially free of,” or “devoid of” such additives.

In particular, according to preferred embodiments of “free of,” “substantially free of,” or “devoid of” active agent protection compounds against light compounds such as piperidinol compounds like tetramethylhydroxypiperidinol citrate, an example of a commercially-available product of such a piperidinol compound is Tinogard® Q available from BASF, which contains 10% active agent, and/or SPF boosters.

A person skilled in the art will take care to select the optional additional additives and/or the amount thereof such that the advantageous properties of the composition according to the disclosure are not, or are not substantially, adversely affected by the envisaged addition.

Needless to say, the composition of the disclosure should be cosmetically or dermatologically acceptable, i.e., it should contain a non-toxic physiologically acceptable. The composition may be in any galenic form normally employed in the cosmetic and dermatological fields which is suitable for topical administration as discussed above.

These auxiliary additives may be present in the composition in a proportion from 0% to 99% (such as from 0.01% to 90%) relative to the total weight of the composition and further such as from 0.1% to 50% (if present), including all ranges and subranges therebetween.

In accordance with the present disclosure, compositions of the present disclosure can be a stand-alone product (for use by itself), or they can be a product for use in conjunction with another composition, for example it can be a basecoat (primer) composition, a color coat composition, or a topcoat (over coat) composition. It should be understood that when compositions of the present disclosure are applied to keratinous materials in the form of any of such compositions, such application can comprise one or more layers of the product. Thus, for example, application of at least one color coat composition can comprise one or more color coat layers; application of the at least topcoat composition can comprise one or more topcoat layers; and application of the at least one basecoat composition can comprise one or more basecoat layers. Preferably, such basecoat, color coat and topcoat compositions contain three or fewer layers of compositions, preferably two or fewer layers of compositions, and preferably a single layer of compositions.

During application of compositions of the present disclosure, basecoat (if present) is typically applied directly to keratinous material, color coat is typically applied either directly to the keratinous material (if no basecoat is present) or to a previously-applied basecoat, and topcoat (if present) is typically applied to a color coat.

According to preferred embodiments of the present disclosure, methods of treating, protecting, enhancing the appearance of, caring for and/or making up keratinous material by applying compositions of the present disclosure to the keratinous material in an amount sufficient to treat, enhance the appearance of, care for and/or make up the keratinous material are provided.

Preferably, “treating” keratinous material includes applying a composition comprising an octinoxate to the keratinous material in an amount sufficient to provide treatment effect to the keratinous material such as, for example, decreasing the appearance of fine lines and/or wrinkles, decreasing the appearance of discoloration (e.g., age spots), increasing smoothness, increasing moisturization, decreasing free radical formation, etc.

Preferably, “protecting” keratinous material includes applying a composition of the present disclosure to the keratinous material in an amount sufficient to protect the keratinous material from damage resulting from exposure to UV rays.

In accordance with the preceding embodiments, compositions of the present disclosure are applied topically to the keratinous material in an amount sufficient to treat, enhance the appearance of, care for and/or make up the keratinous material. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

According to preferred embodiments of the present disclosure, methods of improving photostability of avobenzone and octinoxate (if present), in compositions comprising avobenzone and optionally octinoxate, wherein the methods comprise adding bis-ethylhexyloxyphenol methoxyphenyl triazine to the compositions during formation of the compositions in an amount sufficient to improve photostability of the avobenzone and octinoxate (if present) are provided. Preferably, the bis-ethylhexyloxyphenol methoxyphenyl triazine (BEMT) is added to the compositions in an amount effective to improve photostability of the avobenzone (and optionally octinoxate).

According to preferred embodiments of the present disclosure, methods of making compositions comprising combining bis-ethylhexyloxyphenol methoxyphenyl triazine, avobenzone, and optionally octinoxate in the compositions during formation of the compositions are provided. Preferably, the bis-ethylhexyloxyphenol methoxyphenyl triazine (BEMT) is added to the compositions in an amount effective to improve photostability against light of the avobenzone and octinoxate (if present).

According to preferred embodiments of the present disclosure, methods of improving avobenzone (and optionally improving octinoxate) stability against light of compositions comprising avobenzone and optionally octinoxate, wherein the methods comprise adding bis-ethylhexyloxyphenol methoxyphenyl triazine (BEMT) to the compositions during formation of the compositions in an amount sufficient to improve stability in the compositions against light of the avoboenzone and octinoxate (if present) are provided. Preferably, the bis-ethylhexyloxyphenol methoxyphenyl triazine (BEMT) is added to the compositions in an amount effective to improve stability of the avobenzone (and optionally octinoxate) against light.

According to preferred embodiments of the present disclosure, methods of improving avobenzone stability in compositions which also contain octinoxate, wherein the methods comprise adding bis-ethylhexyloxyphenol methoxyphenyl triazine (BEMT) to the compositions during formation of the compositions in an amount sufficient to improve avobenzone stability in the compositions which also contain octinoxate are provided. Preferably, the bis-ethylhexyloxyphenol methoxyphenyl triazine (BEMT) is added to the compositions in an amount effective to improve stability of the avobenzone against degradation caused by octinoxate.

The present disclosure also envisages kits and/or prepackaged materials suitable for consumer use containing one or more compositions according to the description herein, alone or in combination with other consumer care products such as makeup products such as basecoats, topcoats, removal compositions, etc. The packaging and application device for any subject of the disclosure may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result. The percentages are given on a weight basis.

### Example 1

| INCI US | CREAM SPF50 |
|---|---|
| STEARIC ACID | 2.00 |
| C12-15 ALKYL BENZOATE | 8.00 |
| TOCOPHEROL | 1.00 |
| DIPROPYLENE GLYCOL | 2.11 |
| GLYCERYL STEARATE (and) | 1.50 |
| PEG-100 STEARATE | |
| ALCOHOL DENAT. | 3.00 |
| OCTINOXATE | 5.00 |
| AVOBENZONE | 3.00 |
| GLYCERIN | 4.00 |
| XANTHAN GUM | 0.10 |
| POTASSIUM CETYL | 1.00 |
| PHOSPHATE | |
| CARBOMER | 0.30 |
| TITANIUM DIOXIDE | 1.00 |
| WATER | QS |
| ECAMSULE | 2.00 |
| MICA (and) BARIUM | 3.36 |
| SULFATE (and) TITANIUM | |
| DIOXIDE | |
| CETYL ALCOHOL | 0.70 |
| PROPYLENE GLYCOL | 3.60 |
| TRIETHANOLAMINE | QS |
| CAPRYLYL GLYCOL | 0.30 |
| TROMETHAMINE | QS |
| ACRYLAMIDE/SODIUM | 0.50 |
| ACRYLOYLDIMETHYLTAURATE | |
| COPOLYMER (and) | |
| ISOHEXADECANE (and) | |
| POLYSORBATE 80 | |
| PAEONIA SUFFRUTICOSA | 0.20 |
| ROOT EXTRACT | |
| THERMUS THERMOPHILLUS | 0.10 |
| FERMENT | |
| BEMOTRIZINOL | 6.00 |
| SODIUM COCOYL | 1.00 |
| SARCOSINATE | |
| BUTYLENE GLYCOL (and) | 0.25 |
| BUTYROSPERMUM PARKII | |
| (SHEA) SEEDCAKE | |
| EXTRACT | |
| TRISODIUM | 0.15 |
| ETHYLENEDIAMINE | |
| DISUCCINATE | |
| SQUALANE | 4.50 |
| IRON OXIDES | 0.05 |
| IRON OXIDES | 0.04 |

### Example 2

| INCI US | STICK SPF50 |
|---|---|
| RICINUS COMMUNIS | QS |
| (CASTOR) SEED OIL | |
| TITANIUM DIOXIDE | 0.80 |
| ISOPROPYL PALMITATE | 17.00 |
| BUTYROSPERMUM | 2.00 |
| PARKII (SHEA) BUTTER | |
| OCTINOXATE | 3.00 |
| AVOBENZONE | 3.00 |
| TOCOPHEROL | 1.00 |
| ISOHEXADECANE | 7.00 |
| THEOBROMA CACAO | 2.00 |
| (COCOA) SEED BUTTER | |
| OCTOCRYLENE | 3.50 |
| POLYETHYLENE | 14.00 |
| BEMOTRIZINOL | 5.00 |
| PARAFFIN (and) | 4.00 |
| MICROCRYSTALLINE | |
| WAX (and) SYNTHETIC | |
| WAX | |
| IRON OXIDES | 0.02 |
| IRON OXIDES | 0.01 |

What is claimed is:

1. A composition comprising bemotrizinol, avobenzone, and optionally octinoxate, wherein the bemotrizinol is present in the composition in an amount effective to improve photostability of the avobenzone.

2. The composition of claim 1, wherein the composition comprises octinoxate and the bemotrizinol is present in the composition in an amount effective to improve photostability of the octinoxate.

3. The composition of claim 1, wherein the composition comprises 10% or less by weight relative to the total weight of composition of additional UV filters.

4. The composition of claim 1, wherein the composition comprises 10% or less by weight relative to the total weight of composition of additional organic UV filters.

5. The composition of claim 1, in the form of an emulsion.

6. The composition of claim 1, in the form of a stick.

7. The composition of claim 1, further comprising at least one coloring agent.

8. The composition of claim 1, further comprising at least one active agent.

9. A method of improving avobenzone photostability in a composition comprising avobenzone, comprising adding bis-ethylhexyloxyphenol methoxyphenyl triazine to the composition during formation of the composition in an amount sufficient to improve photostability of the avobenzone.

10. The method of claim 9, wherein the composition further comprises octinoxate and the bemotrizinol is added to the composition in an amount effective to improve photostability of the octinoxate.
